SEQUENCE LISTING (20 sequences)

SEQ ID NO: 1 - DNA, 24 bp, synthetic construct
  source 1..24: mol_type = other DNA
gcatggacgatgcgttgcgtgcac

SEQ ID NO: 2 - DNA, 21 bp, synthetic construct
  source 1..21: mol_type = other DNA
tgatgtttaggtatttaacct

SEQ ID NO: 3 - DNA, 19 bp, synthetic construct
  source 1..19: mol_type = other DNA
cggagtatataaatatccc

SEQ ID NO: 4 - DNA, 19 bp, synthetic construct
  source 1..19: mol_type = other DNA
ctaccaccactcttcttga

SEQ ID NO: 5 - DNA, 55 bp, synthetic construct
  source 1..55: mol_type = other DNA
gtttttatttttatggtgtttaggttaaatacctcttatcatcgataagctagct

SEQ ID NO: 6 - DNA, 56 bp, synthetic construct
  source 1..56: mol_type = other DNA
ttcgggtatagaaaaattgggatatttatatactccggcatttacttataatacag

SEQ ID NO: 7 - DNA, 53 bp, synthetic construct
  source 1..53: mol_type = other DNA
gtttttatttttatggtgtttaggttaaatacctatggcttccaaggtgtacg

SEQ ID NO: 8 - DNA, 59 bp, synthetic construct
  source 1..59: mol_type = other DNA
ttcgggtatagaaaaattgggatatttatatactccgttactgctcgttcttcagcacg

SEQ ID NO: 9 - DNA, 23 bp, synthetic construct
  source 1..23: mol_type = other DNA
ggcggcagaagaagtaacaaagg

SEQ ID NO: 10 - DNA, 23 bp, synthetic construct
  source 1..23: mol_type = other DNA
gcaaatagtcctcttccaacaat

SEQ ID NO: 11 - DNA, 23 bp, synthetic construct
  source 1..23: mol_type = other DNA
agcgcgaccacatggtcctgctg

SEQ ID NO: 12 - DNA, 21 bp, synthetic construct
  source 1..21: mol_type = other DNA
cagccaaccatagcatgcttg

SEQ ID NO: 13 - DNA, 19 bp, synthetic construct
  source 1..19: mol_type = other DNA
atggcttccaaggtgtacg

SEQ ID NO: 14 - DNA, 22 bp, synthetic construct
  source 1..22: mol_type = other DNA
ttactgctcgttcttcagcacg

SEQ ID NO: 15 - DNA, 20 bp, synthetic construct
  source 1..20: mol_type = other DNA
actgcggagcagcttgttat

SEQ ID NO: 16 - DNA, 20 bp, synthetic construct
  source 1..20: mol_type = other DNA
cacaacacgaggtacgggaa

SEQ ID NO: 17 - DNA, 21 bp, synthetic construct
  source 1..21: mol_type = other DNA
gccatccgccccatctaataa

SEQ ID NO: 18 - DNA, 20 bp, synthetic construct
  source 1..20: mol_type = other DNA
ccttccacgagcaaaacacg

SEQ ID NO: 19 - DNA, 20 bp, synthetic construct
  source 1..20: mol_type = other DNA
gagggtggttcgtcacattt

SEQ ID NO: 20 - DNA, 19 bp, synthetic construct
  source 1..19: mol_type = other DNA
taccgccactgcctatgtt

(19) United States
(12) Patent Application Publication
(10) Pub. No.: US 2025/0084424 A1
(43) Pub. Date: Mar. 13, 2025

(54) CHLAMYDOMONAS REINHARDTII CHLOROPLAST-SACCHAROMYCES CEREVISIAE-ESCHERICHIA COLI SHUTTLE VECTOR AND CONSTRUCTION METHOD AND USE THEREOF

(71) Applicant: SHENZHEN UNIVERSITY, Shenzhen, Guangdong (CN)

(72) Inventors: Zhangli HU, Shenzhen (CN); Guiying ZHANG, Shenzhen (CN); Bin JIA, Shenzhen (CN); Chaogang WANG, Shenzhen (CN)

(73) Assignee: SHENZHEN UNIVERSITY, Shenzhen, Guangdong (CN)

(21) Appl. No.: 18/559,882

(22) PCT Filed: Aug. 12, 2022
(86) PCT No.: PCT/CN2022/112289

§ 371 Date: Nov. 9, 2023

(30) Foreign Application Priority Data
Aug. 1, 2022 (CN) 202210916563.X

(51) Int. Cl. C12N 15/81; C12N 15/65; C12N 15/70; C12N 15/82; C12R 1/19; C12R 1/865; C12R 1/89

(52) U.S. Cl. CPC C12N 15/81; C12N 15/65; C12N 15/70; C12N 15/82; C12R 2001/19; C12R 2001/865; C12R 2001/89

(57) ABSTRACT

A Chlamydomonas reinhardtii chloroplast-Saccharomyces cerevisiae-Escherichia coli shuttle vector and a construction method and use thereof. The construction method includes design, chemical synthesis, assembly and identification of a shuttle vector, as well as replication of the shuttle vector in Escherichia coli. The constructed shuttle vector is capable of shuttling and replicating among the Escherichia coli, a yeast, and a Chlamydomonas reinhardtii chloroplast. The shuttle vector of the present invention includes a replication element (CEN) for the yeast, a replication element (ori) for the Escherichia coli, and a Chlamydomonas reinhardtii chloroplast genome (SynCp V1.0). According to the present invention, a totally chemically synthesized shuttle vector is transformed into Chlamydomonas reinhardtii cells, biological functions of the shuttle vector are achieved, a chloroplast genome is extracted and transferred into Escherichia coli, successful replication of the shuttle vector in the Escherichia coli is achieved.

### FIELD

The present invention relates to the fields of synthetic chemistry and molecular biology, in particular, to a Chlamydomonas reinhardtii chloroplast-Saccharomyces cerevisiae-Escherichia coli shuttle vector and a construction method and use thereof.

### BACKGROUND

Chlamydomonas reinhardtii is a single-cell eukaryotic algal model organism, which includes three sets of genetic systems of a nuclear genome, a mitochondrial genome and a chloroplast genome, and is often used to study the mechanism of photosynthesis. With the establishment of genetic transformation technology for the genomes of Chlamydomonas reinhardtii, Chlamydomonas reinhardtii is also widely used as a cell factory to produce recombinant proteins including vaccines, antibodies, drugs, and so on. As a type of microalgae, Chlamydomonas reinhardtii is widely used in the production of biodiesel due to its high carbohydrate and lipid contents, and has broad application prospects. However, the genetic manipulation system of Chlamydomonas reinhardtii is not yet fully mature. Based on existing research results, from the perspective of production of recombinant proteins, the presence of position effect and gene silencing in a nuclear genetic system can lead to lower production of recombinant proteins, thereby limiting the application of the nuclear genetic system. Relative to a cell nucleus, the chloroplast genome is prokaryotic and relatively simple, making a chloroplast become the main research target for production of recombinant proteins. An artificial chloroplast gene of Chlamydomonas reinhardtii has a length of 205 kb, exists in a closed loop manner, contains a total of 99 genes, mainly including related genes involved in photosynthesis, transcription related genes, tRNA and rRNA encoding genes, and the like. In addition to two large-segment repetitive sequences of approximately 22 kb, there are still over 20% of short repetitive sequences distributed between genes in a scattered manner. In addition, the AT content in a chloroplast genome sequence is as high as 65%.

Conventional genetic engineering techniques are merely limited to allowing modifications to existing sequences. Therefore, it will be very meaningful to how to have the ability to shuttle in yeasts, Escherichia coli and Chlamydomonas reinhardtii chloroplasts, and have the ability to significantly alter and arrange genetic content beyond conventional technical abilities. Therefore, there is a demand for synthetic biology, shuttle vector construction and application. However, so far, there is no plasmid that can achieve a shuttle function among yeasts, Escherichia coli, and Chlamydomonas reinhardtii chloroplasts.

Therefore, the prior art still needs improvement.

### SUMMARY

In view of the defects in the above prior art, the present invention provides a Chlamydomonas reinhardtii chloroplast-Saccharomyces cerevisiae-Escherichia coli shuttle vector and a construction method and use thereof, aiming at solving the problem of the lack of plasmids that can achieve a shuttle function among yeasts, Escherichia coli, and Chlamydomonas reinhardtii chloroplasts. According to the present invention, by adopting the method of synthetic biology, by using totally chemically synthesized segments, total chemical de novo synthesis, assembly and replication of the Chlamydomonas reinhardtii chloroplast-Saccharomyces cerevisiae-Escherichia coli shuttle vector are achieved in a yeast-bacterium system, and the provided shuttle vector has an ability to shuttle in yeasts, Escherichia coli, and Chlamydomonas reinhardtii chloroplasts.

The present invention has the following technical solution:

Provided is a Chlamydomonas reinhardtii chloroplast-Saccharomyces cerevisiae-Escherichia coli shuttle vector, wherein a preservation number of the shuttle vector pSSCP is CCTCC M 20221036.

Provided is a construction method for a Chlamydomonas reinhardtii chloroplast-Saccharomyces cerevisiae-Escherichia coli shuttle vector, wherein the construction method includes design, chemical synthesis, assembly and identification of a shuttle vector, as well as replication of the shuttle vector in Escherichia coli, the shuttle vector including a replication element CEN for a yeast, a replication element ori for the Escherichia coli, and a Chlamydomonas reinhardtii chloroplast genome sequence, and the shuttle vector being capable of shuttling and replicating among the Escherichia coli, the yeast, and a Chlamydomonas reinhardtii chloroplast.

According to the construction method for a Chlamydomonas reinhardtii chloroplast-Saccharomyces cerevisiae-Escherichia coli shuttle vector, the shuttle vector further includes a yeast screening marker, an Escherichia coli screening marker, and a Chlamydomonas reinhardtii chloroplast screening marker.

According to the construction method for a Chlamydomonas reinhardtii chloroplast-Saccharomyces cerevisiae-Escherichia coli shuttle vector, the yeast screening marker includes ura-, met-, let-, His-, Ade- or Lys-.

According to the construction method for a Chlamydomonas reinhardtii chloroplast-Saccharomyces cerevisiae-Escherichia coli shuttle vector, the Escherichia coli screening marker includes an ampicillin resistance gene, a kanamycin resistance gene, a gentamicin resistance gene or a tetracycline resistance gene.

According to the construction method for a Chlamydomonas reinhardtii chloroplast-Saccharomyces cerevisiae-Escherichia coli shuttle vector, the Chlamydomonas reinhardtii chloroplast screening marker includes a combination of two or more of a paromomycin resistance gene aphVIII, a streptomycin resistance gene aadA, a hygromycin resistance gene aphVII or a bleomycin resistance gene ble.

Provided is a use of a Chlamydomonas reinhardtii chloroplast-Saccharomyces cerevisiae-Escherichia coli shuttle vector, wherein the Chlamydomonas reinhardtii chloroplast-Saccharomyces cerevisiae-Escherichia coli shuttle vector is used for Chlamydomonas reinhardtii chloroplast engineering modification, the shuttle vector being constructed according to any of the construction methods mentioned above.

According to the use of a Chlamydomonas reinhardtii chloroplast-Saccharomyces cerevisiae-Escherichia coli shuttle vector, a use method of using the shuttle vector for Chlamydomonas reinhardtii chloroplast engineering modification includes the following steps:
8.1 transforming the Chlamydomonas reinhardtii chloroplast-Saccharomyces cerevisiae-Escherichia coli shuttle vector into a yeast;
8.2 carrying out gene editing on the shuttle vector in the yeast;
8.3 extracting and transforming a shuttle vector after gene editing in the yeast into Escherichia coli for vector expanding propagation;
8.4 extracting and transforming a vector after expanding propagation in the Escherichia coli into a Chlamydomonas reinhardtii chloroplast to obtain a Chlamydomonas reinhardtii engineering strain; and
8.5 extracting and transforming a total DNA of the Chlamydomonas reinhardtii engineering strain into the Escherichia coli to obtain a modified new shuttle vector.

According to the use of a Chlamydomonas reinhardtii chloroplast-Saccharomyces cerevisiae-Escherichia coli shuttle vector, the modified new shuttle vector can be transformed into a yeast again for a next round of Chlamydomonas reinhardtii chloroplast genetic engineering modification.

According to the use of a Chlamydomonas reinhardtii chloroplast-Saccharomyces cerevisiae-Escherichia coli shuttle vector, the shuttle vector is used for chloroplast genome editing, chloroplast metabolic pathway design and modification, and chloroplast protein engineering.

Beneficial effects are as follows: the present invention provides a Chlamydomonas reinhardtii chloroplast-Saccharomyces cerevisiae-Escherichia coli shuttle vector and a construction method and use thereof. The construction method includes design, chemical synthesis, assembly and identification of a shuttle vector, as well as replication of the shuttle vector in Escherichia coli. The constructed shuttle vector is capable of shuttling and replicating among the Escherichia coli, a yeast, and a Chlamydomonas reinhardtii chloroplast. The shuttle vector of the present invention includes a replication element (CEN) for the yeast, a replication element (ori) for the Escherichia coli, and a Chlamydomonas reinhardtii chloroplast genome (SynCp V1.0). By adopting the method of synthetic biology, by using totally chemically synthesized segments, total chemical de novo synthesis, assembly and replication of the Chlamydomonas reinhardtii chloroplast-Saccharomyces cerevisiae-Escherichia coli shuttle vector are achieved in a yeast-bacterium system. The totally chemically synthesized shuttle vector is transformed into Chlamydomonas reinhardtii cells, achieving biological functions of the shuttle vector. Finally, a chloroplast genome is extracted and transferred into Escherichia coli, and successful replication of the shuttle vector in Escherichia coli is achieved. According to the present invention, the Chlamydomonas reinhardtii chloroplast-Saccharomyces cerevisiae-Escherichia coli shuttle vector is an efficient platform for carrying out synthetic biology operation, the de novo design, total chemical synthesis, in vitro assembly and identification thereof, assembly and editing in yeast, and replication and editing in Escherichia coli provide a new solution for the rational design, modification and reconstruction of a photosynthetic system of photosynthetic organisms, improvement of photosynthetic efficiency of crops, and solving of agricultural crisis such as food security.

### BRIEF DESCRIPTION OF THE DRAWINGS

FIG. 1 is a map of a plasmid pSSCp provided by an embodiment of the present invention.

FIG. 2 is a schematic diagram of junction PCR results of the plasmid pSSCp provided by an embodiment of the present invention.

FIG. 3 is a schematic diagram of amplification results of aphVIII and BAC sequences of positive transformants of a Chlamydomonas reinhardtii chloroplast provided by an embodiment of the present invention.

FIG. 4 is a schematic diagram of luciferase activity analysis results of CC5168 and partial Chlamydomonas reinhardtii positive transformants in an embodiment of the present invention.

FIG. 5 is a schematic diagram of amplification results of aphVIII, BAC-Seg44 and BAC-Seg1 segments in bacteria of an embodiment of the present invention.

### DETAILED DESCRIPTION OF THE EMBODIMENTS

The present invention provides a Chlamydomonas reinhardtii chloroplast-Saccharomyces cerevisiae-Escherichia coli shuttle vector and a construction method and use thereof. In order to make the purpose, technical solution, and effects of the present invention clearer and more explicit, the present invention is further illustrated in detail below. It should be understood that the specific embodiments described herein are only used to explain the present invention, but not to limit the present invention.

An embodiment of the present invention provides a Chlamydomonas reinhardtii chloroplast-Saccharomyces cerevisiae-Escherichia coli shuttle vector pSSCP, which is preserved the China Center for Type Culture Collection, with a preservation number of CCTCC M 20221036.

An embodiment of the present invention provides a construction method for a Chlamydomonas reinhardtii chloroplast-Saccharomyces cerevisiae-Escherichia coli shuttle vector, and the construction method includes design, chemical synthesis, assembly and identification of a shuttle vector, as well as replication of the shuttle vector in Escherichia coli. The constructed shuttle vector is capable of shuttling and replicating among the Escherichia coli, a yeast, and a Chlamydomonas reinhardtii chloroplast.

In some implementations, the shuttle vector includes a replication element (CEN) for the yeast, a replication element (ori) for the Escherichia coli, and a Chlamydomonas reinhardtii chloroplast genome sequence.

In some specific implementations, the Chlamydomonas reinhardtii chloroplast genome sequence may be a chloroplast genome sequence of wild-type Chlamydomonas reinhardtii, or a redesigned artificial chloroplast genome sequence of Chlamydomonas reinhardtii.

Specifically, during redesign of an artificial chloroplast genome of Chlamydomonas reinhardtii, aphVIII and aadA resistance screening markers are added.

Specifically, the aphVIII resistance screening marker is located at genome 71,780-74,066, and the aadA resistance screening marker is located at genome 176,191-177,820.

The redesigned artificial chloroplast genome of Chlamydomonas reinhardtii has a total of 220,562 bp bases, a 5′atpA-aphVIII-rbcL-3′ expression cassette being located at 71,780-74,066 and a 5′atpA-aadA-rbcL-3′ expression cassette being located at 176,191-177,820 to distinguish from the wild-type chloroplast genome sequence.

In some implementations, the shuttle vector further includes a yeast screening marker, an Escherichia coli screening marker, and a Chlamydomonas reinhardtii chloroplast screening marker.

In some specific implementations, the yeast screening marker can be selected from auxotrophic markers ura-, met-, let-, His-, Ade- or Lys-, preferably ura-.

In some specific implementations, the Escherichia coli screening marker can be selected from an ampicillin resistance gene, a kanamycin resistance gene, a gentamicin resistance gene or a tetracycline resistance gene, etc., preferably the ampicillin resistance gene.

In some specific implementations, the Chlamydomonas reinhardtii chloroplast screening marker can be selected from a combination of two or more of a paromomycin resistance gene aphVIII, a streptomycin resistance gene aadA, a hygromycin resistance gene aphVII or a bleomycin resistance gene ble, preferably a combination of aphVIII and aadA.

The present invention provides implementations and methods for construction of a Saccharomyces cerevisiae-Escherichia coli-Chlamydomonas chloroplast shuttle plasmid. The shuttle plasmid includes a chloroplast genome sequence, and can be replicated in Escherichia coli and yeast, respectively. Specifically, this characteristic of shuttle plasmid can also be used to add a specific metabolic pathway in a shuttle plasmid through yeast homologous recombination.

An embodiment of the present invention further provides a use of a Chlamydomonas reinhardtii chloroplast-Saccharomyces cerevisiae-Escherichia coli shuttle vector, to use the shuttle vector mentioned above for Chlamydomonas reinhardtii chloroplast engineering modification.

In some implementations, the shuttle vector is used for chloroplast genome editing, chloroplast metabolic pathway design and modification, and chloroplast protein engineering.

In a specific example, a luciferase expression gene can be inserted behind a Chlamydomonas reinhardtii paromomycin resistance gene expression cassette to complete editing of a chloroplast genome.

In some implementations, a process of using the shuttle vector for a Chlamydomonas reinhardtii chloroplast to achieve engineering modification of the chloroplast includes the following steps:
S01, transforming the Chlamydomonas reinhardtii chloroplast-Saccharomyces cerevisiae-Escherichia coli shuttle vector into a yeast;
S02, carrying out gene editing on the shuttle vector in the yeast;
S03, extracting and transforming a shuttle vector after gene editing in the yeast into Escherichia coli for vector expanding propagation;
S04, extracting and transforming a vector after expanding propagation in the Escherichia coli into a Chlamydomonas reinhardtii chloroplast to obtain a Chlamydomonas reinhardtii engineering strain; and
S05, extracting and transforming a total DNA of the Chlamydomonas reinhardtii engineering strain into the Escherichia coli to obtain a modified new shuttle vector.

The shuttle vector can replicate and shuttle in a Chlamydomonas reinhardtii chloroplast, Saccharomyces cerevisiae and Escherichia coli. By using its shuttle characteristic, firstly, genetic engineering modification of the vector is carried out in yeast, and then the modified vector is transformed into Chlamydomonas reinhardtii chloroplast to replace the wild chloroplast genome, so as to achieve engineering modification of the chloroplast.

In some implementations, in step S02, the carrying out gene editing on the shuttle vector can include, but is not limited to, chloroplast genome editing, chloroplast metabolic pathway design and modification, or chloroplast protein engineering.

In some implementations, the modified new shuttle vector can be transformed into a yeast again for a next round of Chlamydomonas reinhardtii chloroplast genetic engineering modification. The process of engineering modification is as shown in S01-S05 mentioned above. The method can be used for multiple rounds of recycling, to modify the chloroplast of Chlamydomonas reinhardtii multiple times.

In a specific embodiment, according to the present invention, by using the Chlamydomonas reinhardtii chloroplast-Saccharomyces cerevisiae-Escherichia coli shuttle vector and the use method for the vector to carry out genetic engineering modification on the chloroplast of Chlamydomonas reinhardtii, and by using a luciferase as an example, a Chlamydomonas reinhardtii chloroplast engineering strain that can express the luciferase and has resistance to paromomycin and streptomycin is constructed.

The Chlamydomonas reinhardtii chloroplast-Saccharomyces cerevisiae-Escherichia coli shuttle vector and the construction method and use thereof of the present invention are further explained and illustrated below through specific embodiments.

Unless otherwise specified, materials, reagents, etc. used in the embodiments of the present invention can be purchased commercially; unless otherwise specified, methods, processes, etc. used in the embodiments of the present invention are conventional techniques in the art or can be reasonably set by those skilled in the art based on common knowledge or conventional techniques.

### Embodiment 1 Design of Shuttle Vector

A shuttle plasmid pSSCp included a chloroplast genome sequence of wild-type Chlamydomonas reinhardtii, an Escherichia coli ampicillin resistance gene, Chlamydomonas reinhardtii chloroplast resistance genes aphVIII and aadA, a yeast screening marker ura-, a replication element (CEN) for yeast cells, a replication element (ori) for Escherichia coli, and other elements. Expression cassettes of the chloroplast resistance genes aphVIII and aadA were 5′-atpA-aadA-rbcL-3′ and 5′-atpA-aphVIII-rbcL-3′, respectively.

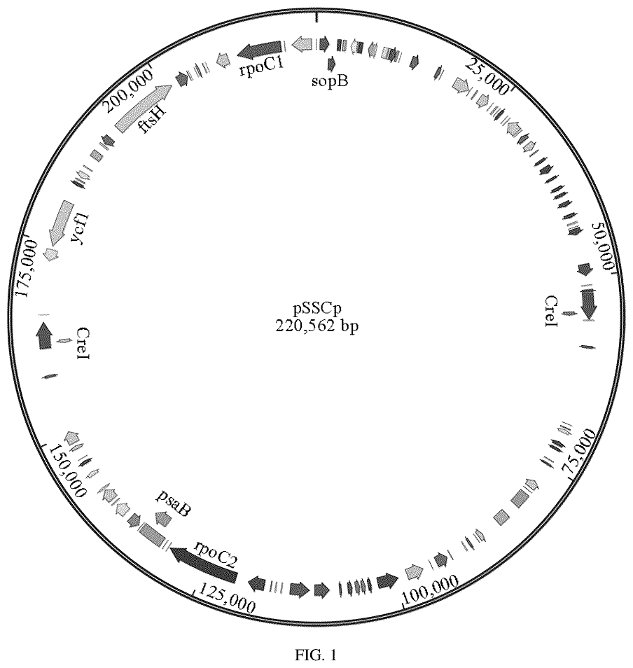

A chloroplast genome sequence (NC_005353.1) of wild-type Chlamydomonas reinhardtii was downloaded from NCBI and designed based on a wild-type chloroplast genome sequence. By taking a chloroplast genome (NC_005353.1) of Chlamydomonas reinhardtii as a basic backbone, a bacterial artificial chromosome (BAC) and a yeast expression vector sequence were inserted at a starting position of the plasmid pSSCp, for replication and stable existence of the plasmid pSSCp in bacteria and yeast. A structure of the plasmid pSSCp is as shown in FIG. 1, with a total length of 220,562 bp, and with features of T7 promoter, sopA, sopB, sopC, cos site, T7 terminator, ampR, CEN6, UAR3, oriV, ori2, repE, from 5′ end to 3′ end. Sequences T7 promoter, sopA, sopB, sopC, cos site, T7 terminator, ori2 and repE are derived from plasmid pBeloBAC11, ampR and CEN6 are derived from plasmid pRS415, UAR3 is derived from plasmid pRS406, and oriV is derived from plasmid pJB866. The above-mentioned plasmids can be purchased commercially.

A 5′atpA-aphVIII-rbcL-3′ resistance gene expression box inserted between sites 71780 and 74066 of the shuttle plasmid, and a 5′-atpA-aadA-rbcL-3′ resistance gene expression box inserted between sites 176191 and 177820 were used as screening markers for the shuttle plasmid during transforming into the chloroplast of Chlamydomonas reinhardtii.

### Embodiment 2 Construction of Shuttle Vector

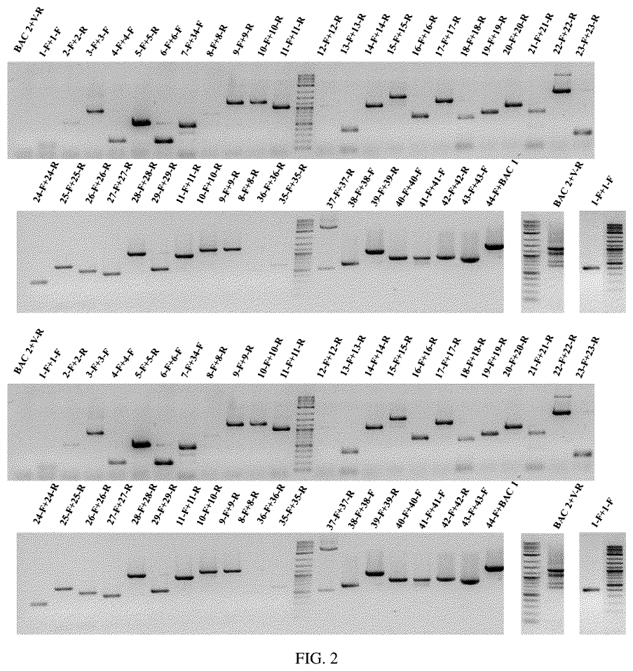

1. The chloroplast genome of Chlamydomonas reinhardtii (excluding BAC and yeast expression vector sequences) designed in Embodiment 1 was divided into 44 primary segments (seg1 to seg44), each of which was approximately 4.9 kb, with 120 bp homologous recombination sequences at both ends. A commercial company was commissioned to synthesize all the primary segments. All the primary segments were connected to a pUC18 vector, could be obtained by a restriction enzyme Not I, and confirmed by sequencing and digestion. The BAC and yeast expression vector sequences were obtained from plasmids pBeloBAC11, pRS415, pRS406 and pJB866 through PCR amplification, respectively. The above-mentioned plasmids could be purchased commercially. There were 120 bp homologous segments between these amplified segments and at the part connected with the chloroplast genome of Chlamydomonas reinhardtii, ensuring homologous recombination between adjacent segments.
2. By using the homologous recombination function of yeast, the above segments were assembled into three intermediate plasmids in yeast. An intermediate plasmid 1 was assembled in yeast BY4741 and included BAC and yeast expression vector sequences as well as designed chloroplast genome segments seg1-7 and seg35-44, and meanwhile, a digestion site I-SceI was inserted between seg7 and 35; an intermediate plasmid 2 was assembled in BY4742 and included designed chloroplast genome segments seg7-seg21, and by using pRS415 as an assembly backbone, a digestion site I-SceI was inserted at two ends of seg7-seg21; an intermediate plasmid 3 was assembled in yeast BY4741 and included designed chloroplast genome segments seg21-seg44, and by using pRS411 as an assembly backbone, a digestion site I-SceI was inserted at two ends of seg21-seg44. The above-mentioned backbone plasmids and yeast strains can be purchased commercially.
3. Through two rounds of yeast hybridization, a yeast strain containing three intermediate plasmids was obtained. Each pair of the three intermediate plasmids had a homologous segment, and one end of the homologous segment contained an I-SceI digestion site. By inducing the expression of I-SceI in yeast cells, the intermediate plasmids 1, 2 and 3 were linearized. With the help of a homologous recombination repair system of yeast cells, the three plasmids were cyclized to obtain a designed circular plasmid pSSCp. The obtained plasmid pSSCp was verified by junction PCR and sequencing, with results as shown in FIG. 2.
4. Expanding propagation and preservation of plasmid pSSCp in Escherichia coli

Saccharomyces cerevisiae containing plasmid pSSCp was cultured overnight in a YPD medium. 4-8 mL of a yeast solution was taken to extract plasmid pSSCp by using a yeast plasmid extraction kit. 0.2-0.4 μg of plasmid DNA was taken to be electrotransformed into Escherichia coli DH5α, and positive transformants, i.e., E. coli DH5α/pSSCp, confirmed by PCR were selected for patent preservation.

In subsequent experiments, the plasmid can directly shuttle into the chloroplast of Chlamydomonas reinhardtii for performing function, or shuttle again into Saccharomyces cerevisiae for editing and modification. In order to prove that the present invention patent can modify the plasmid pSSCp, subsequent examples are carried out in a way of plasmid shuttling yeast, and luciferase genes were inserted by yeast shuttling on the plasmid.

### Embodiment 3 Transformation of Shuttle Vector Into Yeast

### 1. Preparation of Competent Saccharomyces Cerevisiae

After activating and culturing a leu and ura3 gene-deleted yeast on a plate for 2 days, monoclones were selected and inoculated with a YPD liquid for culture at 30° C. and 200 rpm overnight. A culture was taken and inoculated into a 50 mL YPD medium to make a cell concentration to OD 600 being approximately 0.3, and continued to culture for about 5 h until it grew to OD 600 being 1.5-1.6. Cells were collected by centrifugation at 4° C. and 3000 rpm, and resuspended with 20 mL of pre-cooled 1 mol/L sorbitol. Centrifugation was carried out again to collect cells, 16 mL of 1 mol/L of sorbitol, 2 mL of 10×TE and 2 mL of a 1 mol/L lithium acetate solution were added to resuspend cells, and incubation was carried out at 200 rpm at 30° C. for 30 min. After 200 μL of 1 mol/L DTT was added, incubation continued for 15 min. Yeast was collected by centrifugation at 4° C. and 3000 rpm, and then resuspended in 20 mL of pre-cooled 1 mol/L sorbitol, repeated once. After centrifugation again, all liquids were removed, and yeast cells were resuspended with 400 μL of 1 mol/L of sorbitol, and then subpackaged in 100 μL.

2. Electrotransformation of Plasmid pSSCp Into Yeast

2-5 μg of plasmid pSSCp DNA (not exceeding 10 μL) was added into 100 μL of a yeast competent cell, mixed well and then transferred to a 2 mm electroporation cuvette. Electroporation was carried out according to conditions 2.5 kV, 25 μF and 200Ω. After electroporation, 1 mL of sorbitol was added as soon as possible, the cells were transferred to a 15 ml centrifuge tube, and also transferred to a centrifuge tube after the electroporation cuvette was washed with a mixture of 4 mL of sorbitol and YPD (1:1). Cells were collected by centrifugation after 5 h of resuscitation of the centrifuge tube at 30° C. and 200 rpm. After washed with sterile water 1-2 times, the cells were resuspended with 4 mL of an SC-let medium and then cultured at 30° C. and 200 rpm for 24 h. Then, according to the concentration of a bacterial solution, 10-200 μL of cells were selected to coat an SC-let solid medium. Culture was carried out at 30° C. for 1-2 days until colonies grew. Some colonies were randomly selected, and all adaptor sequences were detected by using PCR. Colonies whose adaptors could all be detected were considered as positive transformants.

### Embodiment 4 Editing and Modification of Shuttle Vector in Yeast

By using the homologous recombination characteristic of yeast, plasmid pSSCp can be edited and modified, so that it can be appropriate for various subsequent experimental needs. In this embodiment, by taking a luciferase as an example, it was inserted into 74067 bp of plasmid pSSCp, so that it was located behind an expression cassette of a paromomycin resistance gene of Chlamydomonas reinhardtii. Unless otherwise specified, the plasmids and strains can be purchased commercially.

### 1. Construction of Yeast Resistance Homologous Recombination Unit

Gene ura3 was selected as a screening marker to design a homologous recombination unit. A DNA sequence of approximately 1 kB at 73000 bp-74066 bp on a plasmid pSSCp was amplified with PCR to serve as a homologous left arm. Primer sequences are shown in SEQ. ID NO.1-SEQ. ID NO.2:

SEQ. ID NO. 1: LF:
gcatggacgatgcgttgcgtgcac
SEQ. ID NO. 2: LR:
tgatgtttaggtatttaacct

Annealing for amplification was carried out at a temperature of 58° C., cycled 25 times. A DNA sequence of approximately 1 kB at 74066 bp-75066 bp on a plasmid pSSCp was amplified with PCR to serve as a homologous right arm. A PCR product was purified by gel slices. Primers thereof are shown in SEQ. ID NO.3-SEQ. ID NO.4:

SEQ. ID NO. 3: RF:
cggagtatataaatatccc,
SEQ. ID NO. 4: RR:
ctaccaccactcttcttga.

Annealing for amplification was carried out at a temperature of 56° C., cycled 25 times. A PCR product was purified by gel slices. ura3 (approximately 1.1 kb) was amplified from a plasmid pYES2 by using PCR, and during amplification, 30 bp sequences homologous to the homologous left and right arms were added to the primers, respectively. Primers are shown in SEQ. ID NO.5-SEQ. ID NO.6:

SEQ. ID NO. 5: UraF:
gtttttatttttatggtgtttaggttaaa
tacctcttatcatcgataagctagct
SEQ. ID NO. 6: UraR:
ttcgggtatagaaaaattgggatatttat
atactccggcatttacttataatacag.

Stabilizing annealing was carried out at 54° C., cycled 25 times. A PCR product was purified by gel slices. 3 PCR segments were assembled into an approximately 3.1 kb segment by using overlap PCR. Primers LF and RR were used, homologous left arm DNA was used as a template, and homologous right arm DNA and ura3 DNA were of 100 ng each. Annealing was carried out at a temperature of 52° C., cycled 25 times. Therefore, a resistance homologous recombination unit was obtained.

### 2. Construction of Luciferase Homologous Recombination Unit

The recombinant unit had the same homologous left arm and right arm as those of the resistance homologous recombination unit. By using a plasmid psaD-luc as a template, a luciferase expression cassette was amplified, and during amplification, 30 bp sequences homologous to the homologous left and right arms were added to the primers, respectively. Primers thereof are shown in SEQ. ID NO.7-SEQ. ID NO.8:

SEQ. ID NO. 7: LucF:
gtttttatttttatggtgtttaggtta
aatacctatggcttccaaggtgtacg,
SEQ. ID NO. 8: LucR:
ttcgggtatagaaaaattgggatatttat
atactccgttactgctcgttcttcagcacg.

Amplification conditions were as follows: annealing was carried out at a temperature of 52° C. cycled 25 times. A PCR product was purified by gel slices. A left arm segment, a right arm segment and a luciferase segment were assembled into an approximately 3 kb segment by using overlap PCR to obtain a luciferase homologous recombination unit. Primers LF and RR were used, homologous left arm DNA was used as a template, and homologous right arm DNA and DNA of the luciferase expression cassette were of 100 ng each.
3. Editing and Modification of Plasmid pSSCP
(1) After a yeast containing plasmid pSSCP was taken and inoculated with a YPD liquid for culture, competent cells were prepared according to the method in Embodiment 2. After 2-5 μg of resistance homologous recombination unit DNA was taken and mixed with 100 μL of the competent cells, electrotransformation was carried out according to the method in Embodiment 2. After the electrotransformation was completed, 10-200 μL of cells were taken to be coated on a plate containing SD-ura and cultured at 30° C. for 1-2 days until colonies grew. Some colonies were randomly selected, and firstly, ura gene was detected by using PCR. Primer sequences are shown in SEQ. ID NO.9-SEQ. ID NO.10:
SEQ. ID NO.9: UraTF: ggcggcagaagaagtaacaaagg
SEQ. ID NO.10: UraTR: gcaaatagtcctcttccaacaat. A target segment is approximately 400 bp. Then, homologous recombination primers were used to detect transformants with ura positive, and sequences thereof are shown in SEQ. ID NO.11-SEQ. ID NO.12:
SEQ. ID NO.11: HRTF: agcgcgaccacatggtcctgctg
SEQ. ID NO.12: HRTR: cagccaaccatagcatgcttg. For positive yeast transformants subjected to homologous recombination replacement, approximately 3.1 kb of segments could be detected, and for transformants without editing, approximately 2 kb of segments could be detected. The transformants with ura and homologous recombination detection positive were selected for second transformation.
(2) The transformants of (1) were taken and inoculated with YPD to prepare competent cells. After 2-5 μg of luciferase homologous recombination unit DNA was taken, and 100 μL of the competent cells were added for mixing, electrotransformation was carried out according to the method in Embodiment 2. After the electrotransformation was completed, 10-200 μL of cells were taken to be coated on a complete SD solid medium containing 0.1 g/mL 5-Fluoroorotic acid (5-FOA) with pH 4, until colonies grew. Some colonies were randomly selected, and luciferase gene was detected by PCR, and sequences thereof are shown in SEQ. ID NO.13-SEQ. ID NO.14:

SEQ. ID NO. 13: LucTF:
atggcttccaaggtgtacg
SEQ. ID NO. 14: LucTR:
ttactgctcgttcttcagcacg.

Then, recombinant detection primers HRTF and HRTR were used for detection. For positive yeast transformants subjected to homologous recombination replacement, approximately 2.8 kb of segments could be detected, and for transformants without editing, approximately 3.1 kb of segments could be detected. The required transformant was the one that has been detected positive for both rounds of PCR, and contained a successfully edited plasmid pSSCPE1.

### Embodiment 5 Shuttling and Replication of Shuttle Vector From Yeast to Escherichia Coli

1. Extraction of Total DNA of Yeast Plasmid pSSCPE1

2-4 mL of the Saccharomyces cerevisiae containing plasmid pSSCPE1 in Embodiment 4 was taken and centrifuged to collect cells, 500 μL of ddH₂O was added for washing 1-2 times, and a supernatant was carefully removed by using a vacuum pump. 100 μL of yeast breaking buffer (50 mM Tris (pH8.0), 100 mM NaCl, 1% SDS, 2% Triton X-100, 1 mM EDTA), 100 μL of 0.5 mm pickled glass beads and 200 μL of a phenol chloroform solution (25:24) were added, mixed and then subjected to intense vortex shaking for 10 min. After 100 μL of ddH₂O was added and inverted several times, centrifugation was carried out at 12000 rpm for 10 min. A supernatant water phase was carefully sucked and transferred to a new centrifuge tube, 500 μL of 100% ethanol was added, and the new centrifuge tube was put at −20° C. for precipitation for 30 min. Centrifugation was carried out at 13000 rpm for 5 min to collect precipitated DNA. After a precipitate was washed 1-2 times with 500 μL of 70% ethanol, an appropriate amount of ddH₂O was taken to dissolve DNA.

2. Electrotransformation of Total DNA into Escherichia Coli
1) Overnight cultured and activated Escherichia coli DH5α was taken and inoculated in 1% into a 50 mL LB medium for culture at 37° C. and 200 rpm until OD 600 was 0.5-0.6. Transfer was carried out to a pre-cooled 50 mL centrifuge tube, and shock cooling was carried out rapidly in an ice water mixture for 15 min. Centrifugation was carried out at 4° C. and 4000 rpm for 10 min to collect bacterial cells. The bacterial cells were washed 2 times with 50 mL of pre-cooled ddH₂O. Then, after the bacterial cells were washed 2 times with 10% glycerol, and cells were resuspended with 200-250 μL of 10% glycerol. The cells were subpackaged according to a volume of 100 μL to obtain competent cells.
2) 1-2 μg of the total DNA extracted in step 1 was taken and added into 50 μL of the competent cells, incubated on ice for 5 min and then transferred to a pre-cooled 2 mm electroporation cuvette for ice bath for 10 min. Electroporation was carried out according to conditions of 2.5 kV, 200Ω and 25 μF. After the electroporation was completed, 1 mL of a 37° C. LB medium was added, and a mixed solution was transferred into a 1.5 mL centrifuge tube. Shake culture was carried out at 37° C. and 220 rpm for 1 h. Then coated on an LB solid plate containing 100 μg/mL DH5α, and cultured overnight at 37° C., until colonies grew. Some colonies were randomly selected, and all adaptor sequences were detected by using PCR. Colonies whose adaptors could all be detected were considered as positive transformants, and the transformants contained plasmid pSSCPE1.

### Embodiment 6 Shuttling of Shuttle Vector From Escherichia Coli to Chlamydomonas Reinhardtii Chloroplast

### 1. Transfer of Chlamydomonas Reinhardtii Chloroplast Shuttle Plasmid Into Chlamydomonas Reinhardtii Chloroplast

### 1) Preparation of Gold Powder

30 mg of gold powder with a diameter of 1 μm was weighed and placed in a 1.5 mL centrifuge tube. 1 mL of 70% ethanol was added. Vigorous shake washing was carried out for 5 min. Soaking was carried out for 15 min. Centrifugation was carried out at 4000 rpm for 5 s, and a supernatant was sucked and discarded. 1 mL of ddH₂O was added. Vigorous shake vortex was carried out for 1 min. Standing was carried out for 1 min, and centrifugation was carried out at 4000 rpm for 5 min. Repeated washing was carried out 3 times. 500 μL of 50% glycerol was added to a gold powder precipitate, and the gold powder precipitate was stored at 4° C. for standby use.

### 2) Envelope of Gold Powder

The gold powder stored in the 50% glycerol was fully shaken for 5 min, and 50 μL of the gold powder was taken and placed into a 1.5 mL centrifuge tube. 5 μg of a plasmid, 50 μL of CaCl₂ and 20 μL of 0.1 M spermidine were added at a time. Full vortex shaking was carried out for 3 min. Standing was carried out for 1 min. Centrifugation was carried out at 4000 rpm for 5 s. A supernatant was sucked and discarded, sufficient 70% ethanol was added for rinsing, and a supernatant was sucked and discarded, repeated twice. After rinsing, sufficient anhydrous ethanol was added for rinsing, and a supernatant was sucked and discarded. An appropriate amount of anhydrous ethanol was added, and quick vortex was carried out for 5 s to resuspend a precipitate.

### 3) Biolistic Transformation

A receptor algal cell (CC5168) cultured to a cell concentration of 2×10⁶ cell/mL was taken, collected by centrifugation (3500×g, 5 min), and resuspended with a fresh TAP medium to 1×10⁸ cell/mL. 250 μL of a resuspended algal cell was taken, laid at a center of a 90×15 mm screening plate, and dried for 2 hours in dark at a room temperature.

Preparation for bombardment DNA particles: 50 μL of gold powder was taken, add 5.0 μL of pSSCPE1 DNA (1.0 μg/μL), 50 μL of 2.5 M CaCl₂, and 20 μL of 0.1 mM spermidine were added in sequence, shaking for mixing well was carried out, centrifugation was carried out, washing was carried out with 70% alcohol, and finally resuspending was carried out with 100 μL of anhydrous ethanol, taking 10 μL for bombardment each time. A gene gun was Biolistic PDS-1000/He Particle Delivery System (BIO-RAD), with the following bombardment parameters: a crackable film 1100 psi, and a bombardment distance of 9 cm. After bombardment was completed, a plate was cultured in dark at 25° C. for 24 hours, and then transferred into an incubator at 25° C. with a photoperiodic ratio of 16:8 and continued to culture for 3-4 weeks until green monoclones grew.

### 2. Identification of Transformants

After green transformants grew, in a super clean bench, the transformants were streaked with a toothpick to a new streptomycin resistance (150 μg/mL) culture dish, and continued to culture. Chlamydomonas reinhardtii monoclone cells were picked up to 50 μL of chelex. After shaking and mixing well, at 98° C. for 30 min, immediate cooling was carried out on ice. Shaking for mixing well and centrifugation were carried out. By using a supernatant as a template, PCR amplification was carried out with a primer pair of aphVIII-F and aphVIII-R, and positive transformants were subjected to primary screening. The primers are shown in SEQ. ID NO.15-SEQ. ID NO.16:

SEQ. ID NO. 15: aph VIII-F:
actgcggagcagcttgttat
SEQ. ID NO. 16: aph VIII-R:
cacaacacgaggtacgggaa

Subsequently, a genome of the transformants subjected to primary screening positive was extracted with M5 Plant Genomic DNA Max Kit (Cat. MF136-01, Beijing). By using the genome as a template, PCR amplification was carried out with a primer pair of BAC44-F and BAC44-R and a primer pair of BAC1-F and BAC1-R, respectively. The primers are shown in SEQ. ID NO.17-SEQ. ID NO.20:

SEQ. ID NO. 17: BAC44-F:
gccatccgccccatctaataa
SEQ. ID NO. 18: BAC44-R:
ccttccacgagcaaaacacg
SEQ. ID NO. 19: BAC1-F:
gagggtggttcgtcacattt
SEQ. ID NO. 20: BAC1-R:
taccgccactgcctatgtt

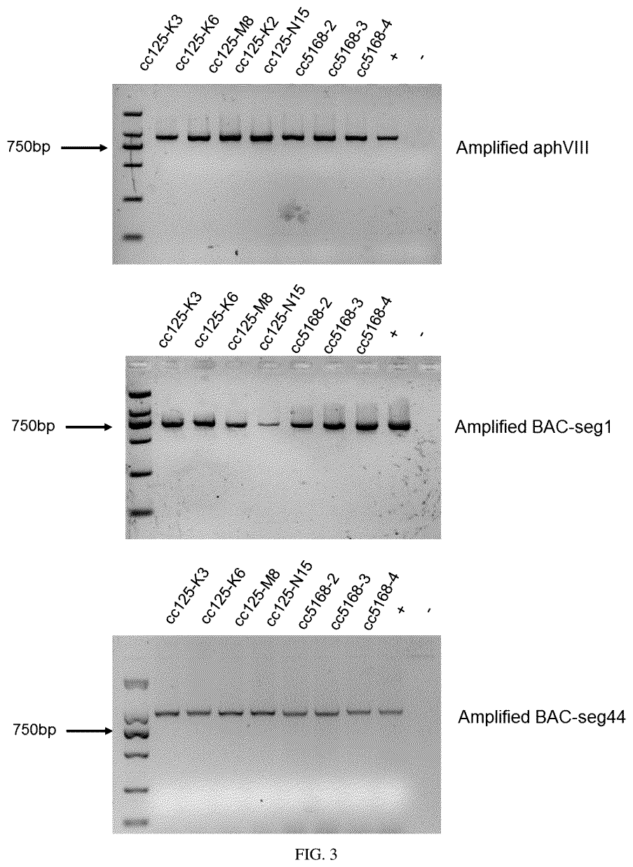

A PCR product was subjected to 1.2% agarose gel electrophoresis for further positive identification to obtain transformants containing both aphVIII and BAC sequences (as shown in FIG. 3), and these transformants were positive transformants of Chlamydomonas reinhardtii chloroplasts.

### Embodiment 7 Function Analysis of Positive Transformants of Chlamydomonas Reinhardtii Chloroplasts

### Luciferase Activity Analysis

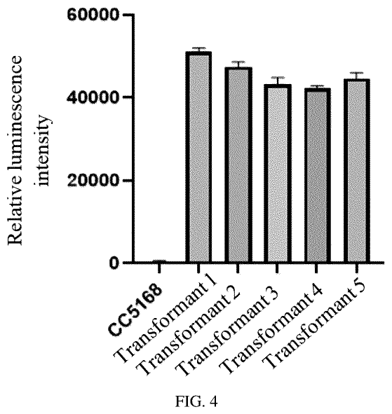

Part of the positive transformants of Chlamydomonas reinhardtii chloroplasts were taken and respectively inoculated to a TAP liquid medium, and grew under conditions of 25° C., 30 μE/m²/s, 16 h of light, and 8 h of darkness until the middle to late logarithmic phase, with a cell number of 3×10⁶-6×10⁶. Wild algae CC5168 was inoculated to a TAP liquid medium and then cultured in dark for 6-8 days. 2-4 mL of algae cells were collected by centrifugation at 12000 g, 0.5 mL of Tris-EDTA buffer (pH7.8, 10 mM Tris, 1 mM EDTA) and 0.3 g of glass beads were added, and high-speed shaking was carried out for 10 min at 4° C. in a grinder. 50 μL of a lysate was taken, 140 μL of a reaction buffer (0.1 M K₂HPO₄, 0.5 M NaCl and 1 mM EDTA, pH to 7.8) and 10 μL of coelenterazine were added for dark culture for 5 min at a room temperature; a reaction solution was added into a black opaque 96-well plate, and integration time was set to be 1 s in a chemiluminescence microplate reader to detect chemiluminescence intensity. Results are as shown in FIG. 4, the relative luminescence intensity of chloroplast positive transformants is significantly higher than that of wild-type CC5168, and the relative luminescence intensity of the transformants is 80-100 times that of CC5168, indicating that the luciferase gene in the positive transformants of Chlamydomonas reinhardtii chloroplasts can be efficiently expressed.

### Embodiment 8 Shuttling and Replication of Shuttle Vector From Chlamydomonas Reinhardtii Chloroplast to Escherichia Coli

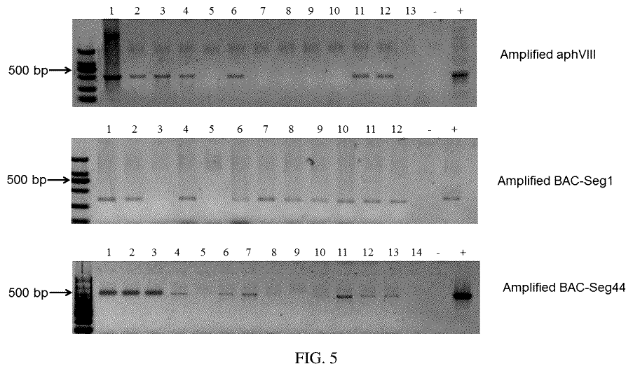

A total DNA of transformants of Chlamydomonas reinhardtii was extracted and transformed again into Escherichia coli. Through resistance screening, 20 single transformants were obtained. Plasmid DNA of Escherichia coli transformants was extracted for PCR amplification identification. A primer pair of aphVIII-F and aphVIII-R, a primer pair of BAC44-F and BAC44-R, and a primer pair of BAC1-F and BAC1-R were used to amplify exogenous segments such as aphVIII, BAC-Seg44 and BAC-Seg1, respectively (results were as shown in FIG. 5), wherein 6 transformants were identified to contain complete shuttle plasmids. In a control experiment, the genome DNA of wild-type Chlamydomonas reinhardtii CC125 was transformed into Escherichia coli without obtaining any transformants. The results indicate that the plasmid can shuttle in Chlamydomonas reinhardtii chloroplasts, Escherichia coli and Saccharomyces cerevisiae. The 6 complete shuttle plasmids obtained in this embodiment can be subjected to editing and modification again according to the methods described in Embodiments 3-8. The above embodiments show that the plasmid provided by the present invention can efficiently carry out modification and function upgrade on Chlamydomonas chloroplast genome, truly building a synthetic biology platform of “design-build-test cycle” of chloroplast genomes of photosynthetic organism.

In summary, the present invention provides a Chlamydomonas reinhardtii chloroplast-Saccharomyces cerevisiae-Escherichia coli shuttle vector and a construction method and use thereof. The construction method includes design, chemical synthesis, assembly and identification of a shuttle vector, as well as replication of the shuttle vector in Escherichia coli. The constructed shuttle vector is capable of shuttling and replicating among the Escherichia coli, a yeast, and a Chlamydomonas reinhardtii chloroplast. The shuttle vector of the present invention includes a replication element (CEN) for the yeast, a replication element (ori) for the Escherichia coli, and a Chlamydomonas reinhardtii chloroplast genome (SynCp V1.0). By adopting the method of synthetic biology, by using totally chemically synthesized segments, total chemical de novo synthesis, assembly and replication of the Chlamydomonas reinhardtii chloroplast-Saccharomyces cerevisiae-Escherichia coli shuttle vector are achieved in a yeast-bacterium system. The totally chemically synthesized shuttle vector is transformed into Chlamydomonas reinhardtii cells, achieving biological functions of the shuttle vector. Finally, a chloroplast genome is extracted and transferred into Escherichia coli, and successful replication of the shuttle vector in Escherichia coli is achieved. According to the present invention, the Chlamydomonas reinhardtii chloroplast-Saccharomyces cerevisiae-Escherichia coli shuttle vector is an efficient platform for carrying out synthetic biology operation, the de novo design, total chemical synthesis, in vitro assembly and identification thereof, assembly and editing in yeast, and replication and editing in Escherichia coli provide a new solution for the rational design, modification and reconstruction of a photosynthetic system of photosynthetic organisms, improvement of photosynthetic efficiency of crops, and solving of agricultural crisis such as food security.

It should be understood that the application of the present invention is not limited to the above examples. For those of ordinary skill in the art, improvements or transformations can be made according to the above description, and all these improvements and transformations should fall within the protection scope of the claims attached to the present invention.

1-10. (canceled)

11. A Chlamydomonas reinhardtii chloroplast-Saccharomyces cerevisiae-Escherichia coli shuttle vector, wherein a preservation number of the shuttle vector pSSCP is CCTCC M 20221036.

12. A construction method for a Chlamydomonas reinhardtii chloroplast-Saccharomyces cerevisiae-Escherichia coli shuttle vector, the construction method comprising design, chemical synthesis, assembly and identification of a shuttle vector, as well as replication of the shuttle vector in Escherichia coli, the shuttle vector comprising a replication element CEN for a yeast, a replication element ori for the Escherichia coli, and a Chlamydomonas reinhardtii chloroplast genome sequence, and the shuttle vector being capable of shuttling and replicating among the Escherichia coli, the yeast, and a Chlamydomonas reinhardtii chloroplast.

13. The construction method for a Chlamydomonas reinhardtii chloroplast-Saccharomyces cerevisiae-Escherichia coli shuttle vector according to claim 12, wherein the shuttle vector further comprises a yeast screening marker, an Escherichia coli screening marker, and a Chlamydomonas reinhardtii chloroplast screening marker.

14. The construction method for a Chlamydomonas reinhardtii chloroplast-Saccharomyces cerevisiae-Escherichia coli shuttle vector according to claim 13, wherein the yeast screening marker comprises ura-, met-, let-, His-, Ade- or Lys-.

15. The construction method for a Chlamydomonas reinhardtii chloroplast-Saccharomyces cerevisiae-Escherichia coli shuttle vector according to claim 13, wherein the Escherichia coli screening marker comprises an ampicillin resistance gene, a kanamycin resistance gene, a gentamicin resistance gene or a tetracycline resistance gene.

16. The construction method for a Chlamydomonas reinhardtii chloroplast-Saccharomyces cerevisiae-Escherichia coli shuttle vector according to claim 13, wherein the Chlamydomonas reinhardtii chloroplast screening marker comprises a combination of two or more of a paromomycin resistance gene aphVIII, a streptomycin resistance gene aadA, a hygromycin resistance gene aphVII or a bleomycin resistance gene ble.

17. A use of a Chlamydomonas reinhardtii chloroplast-Saccharomyces cerevisiae-Escherichia coli shuttle vector, wherein the Chlamydomonas reinhardtii chloroplast-Saccharomyces cerevisiae-Escherichia coli shuttle vector is used for Chlamydomonas reinhardtii chloroplast engineering modification, the shuttle vector being constructed according to the construction method of claim 12.

18. The use of a Chlamydomonas reinhardtii chloroplast-Saccharomyces cerevisiae-Escherichia coli shuttle vector according to claim 17, wherein a use method of using the shuttle vector for Chlamydomonas reinhardtii chloroplast engineering modification comprises the following steps:
18.1 transforming the Chlamydomonas reinhardtii chloroplast-Saccharomyces cerevisiae-Escherichia coli shuttle vector into a yeast;
18.2 carrying out gene editing on the shuttle vector in the yeast;
18.3 extracting and transforming a shuttle vector after gene editing in the yeast into Escherichia coli for vector expanding propagation;
18.4 extracting and transforming a vector after expanding propagation in the Escherichia coli into a Chlamydomonas reinhardtii chloroplast to obtain a Chlamydomonas reinhardtii engineering strain; and
18.5 extracting and transforming a total DNA of the Chlamydomonas reinhardtii engineering strain into the Escherichia coli to obtain a modified new shuttle vector.

19. The use of a Chlamydomonas reinhardtii chloroplast-Saccharomyces cerevisiae-Escherichia coli shuttle vector according to claim 18, wherein the modified new shuttle vector can be transformed into a yeast again for a next round of Chlamydomonas reinhardtii chloroplast genetic engineering modification.

20. The use of a Chlamydomonas reinhardtii chloroplast-Saccharomyces cerevisiae-Escherichia coli shuttle vector according to claim 17, wherein the shuttle vector is used for chloroplast genome editing, chloroplast metabolic pathway design and modification, and chloroplast protein engineering.
